(19) United States
(12) Patent Application Publication
(10) Pub. No.: US 2008/0090893 A1
(43) Pub. Date: Apr. 17, 2008

(54) Medicinal Composition for Prevention of Transition to Operative Treatment for Prostatic Hypertrophy

(75) Inventors: Yoshio Okubo, Tokyo (JP); Tomoji Shimizu, Tokyo (JP); Nobuhiko Arai, Tokyo (JP); Yasuhiro Omori, Tokyo (JP)

Correspondence Address: SUGHRUE MION, PLLC, 2100 PENNSYLVANIA AVENUE, N.W., SUITE 800, WASHINGTON, DC, 20037, US

(73) Assignee: Kissei Pharmaceutial Co., Ltd., Matsumoto-shi (JP)

(21) Appl. No.: 11/576,800

(22) PCT Filed: Oct. 4, 2005
(86) PCT No.: PCT/JP05/18356

§ 371 Date: Apr. 5, 2007

(30) Foreign Application Priority Data
Oct. 6, 2004 (JP) 2004-294358
Jun. 1, 2005 (JP) 2005-160843

(51) Int. Cl. A61K 31/404; A61P 35/00

(52) U.S. Cl. 514/415

(57) ABSTRACT

The present invention provides an agent useful for the prevention of transition to surgical therapy for benign prostatic hyperplasia and the like. The present invention relates to a pharmaceutical composition for the prevention of transition to surgical therapy for benign prostatic hyperplasia, which comprises an indoline derivative represented by the following general formula (I) or a pharmaceutically acceptable salt thereof (in the formula, R represents an optionally substituted aliphatic acyl group, a hydroxyalkyl group, an aliphatic acyloxyalkyl group, a substituted lower alkyl group, an optionally substituted aromatic acyl group, a furoyl group, a pyridylcarbonyl group or the like; R¹ represents a cyano group or a carbamoyl group; and R² represents an optionally substituted lower alkyl group).

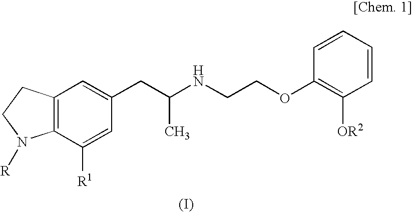

### TECHNICAL FIELD

The present invention relates to a pharmaceutical composition for the prevention of transition to surgical therapy for benign prostatic hyperplasia.

More particularly, the present invention relates to a pharmaceutical composition for the prevention of transition to surgical therapy for benign prostatic hyperplasia, which comprises an indoline derivative represented by a general formula:

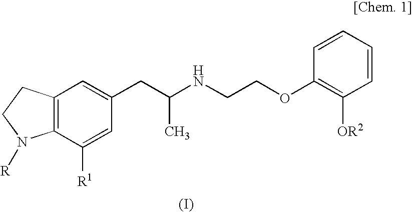

in the formula, R represents an aliphatic acyl group which may have one or more of a halogen atom, a hydroxyl group, a lower alkoxy group, a carboxyl group, a lower alkoxy-carbonyl group, a cycloalkyl group or an aryl group as a substituent group and may have an unsaturated bond in some cases, a hydroxyalkyl group, an aliphatic acyloxyalkyl group, a lower alkyl group which has a lower alkoxy group, a carboxy group, a lower alkoxycarbonyl group, an aryl-substituted lower alkoxycarbonyl group, a carbamoyl group, a mono or dialkyl-substituted carbamoyl group or a cyano group as a substituent group, an aromatic acyl group which may have one or more halogen atoms as a substituent group, a furoyl group or a pyridylcarbonyl group, R¹ represents a cyano group or a carbamoyl group; and R² represents a lower alkyl group which may have one or more of a halogen atom, a cyano group or an aryl group as a substituent group, or a pharmaceutically acceptable salt thereof as an active ingredient, and the like.

### BACKGROUND ART

Benign prostatic hyperplasia is a disease which has high generation frequency in aged males, and it is said that 70% of males of sixties are suffering from this. In the aging society of recent years, the number of patients of benign prostatic hyperplasia has been increasing and drawing great attention.

Main therapeutic methods for benign prostatic hyperplasia are drug therapy and surgical therapy. As the surgical therapies, open prostatectomy, transurethral resection of the prostate (TUR-P), thermotherapy, laser therapy, stent indwelling method and the like are known. The open prostatectomy becomes indication in the case of large size of prostate, in the case of repeated urinary retention and the like, but is most invasive. TUR-P is a method in which an endoscope is inserted from urethra, and the prostate is cut off with an electric surgical knife from the inside while observing the prostate using a mirror. Currently, this is the most popularized standard method as the surgical therapy for benign prostatic hyperplasia, which is carried out on about fifty thousand cases a year in Japan and about three hundred thousand cases a year in the United States. In comparison with the open prostatectomy, bleeding is little, and recovery after the operation is quick. Also, the thermotherapy is a method in which opening of the urethra is improved by destroying tissue of the prostatic inner gland by heating them through the irradiation of microwave from the urethra. This is a therapeutic method which can be carried out for out-patients, causes little pain and is effective on the benign prostatic hyperplasia of moderate or less severity. The laser therapy is a method in which the tissue of prostatic hypertrophy adenoma is allowed to undergo necrosis or is destroyed by irradiating laser. Since the method has many advantages in comparison with TUR-P, such as little bleeding, short treating period of time, little pain in patients and the like, this is drawing attention as a highly advanced therapeutic method ranking with the thermotherapy. The stent indwelling method is a method in which a duct (stent) is indwelled in a urethral part thinned by the pressure of swollen prostate (e.g., see Non-patent Reference 1)

As the drug therapy for benign prostatic hyperplasia, an α1 adrenoceptor antagonist, an anti-androgen drug, a herb medicine and the like are used. It has been reported that the effect of an al adrenoceptor antagonist, tamsulosin hydrochloride, to reduce storage disorder is indicated to be similar to that of TUR-P in short terms (e.g., see Non-patent Reference 2).

In deciding the therapeutic method, in general, overall severity based on I-PSS (International Prostate Symptom Score) total score, QOL score, maximum flow rate, residual urine volume and prostate volume is used. For example, when the overall severity is moderate or more, surgical therapy such as the TUR-P or the like become the indication (e.g., Non-patent References 3 and 4). However, since there are many patients who do not desire the surgical therapy as an invasive treatment, the development of a drug which can prevent the transition to a surgical therapy in severe patients who are subject to the surgical therapy has been desired.

The indoline derivative represented by the above general formula (I) or a pharmaceutically acceptable salt thereof is markedly useful as an agent for the treatment of dysuria associated with benign prostatic hyperplasia and the like, because it has a selective inhibitory activity against urethral smooth muscle contraction and can lower urethral pressure without great influence upon blood pressure (see Patent References 1 to 3). However, there are no descriptions or suggestions therein that the above indoline derivatives (I) or pharmaceutically acceptable salts thereof, particularly a compound represented by the following formula (II) (generic name: silodosin), can prevent the transition of a patient with benign prostatic hyperplasia who is subject to surgical therapy to the surgical therapy.

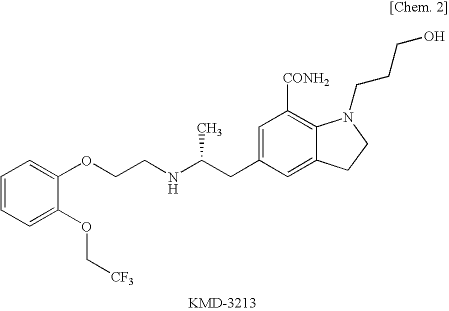

Patent Reference 1: JP-A-H06-220015;

Patent Reference 2: International Publication No. 99-15202 pamphlet;

Patent Reference 3: International Publication No. 2004-22538 pamphlet;

Non-patent Reference 1: Satoru Takahashi, “Yoku Wakaru Zenritsusen No Byoki (Good Understanding on Prostatic Diseases)”, published by Iwanami Shoten, Mar. 4, 2004, pp. 58-94;

Non-patent Reference 2: B. Djavan, Shinyaku To Chiryo (New Drugs and Treatments), No. 442, vol. 53, no. 3, 2003, pp. 11-12;

Non-patent Reference 3: Hainyo Shogai Rinsho Shiken Gaidolain Sakusei Iinkai Hen (Edited by Urinary Disturbance Clinical Test Guideline Preparation Committee), “Hainyo Shogai Rinsho Shiken Gaidolain Daiichibu Zenritsusen HidaiSho (Urinary Disturbance Clinical Test Guideline Part 1, Benign Prostatic Hyperplasia”, published by Igaku Tosho Shuppan, 1997;

Non-patent Reference 4: Hinyokika Ryoiki No Chiryo Hyojunka Ni Kansuru Kenkyu Han Hen (Edited by a Research Group on the Treatment Standardization in the Field of Urology), “Zenritsusen Hidaisho Shinryo Gaidolain (Guideline on the Medical Examination and Treatment of Benign Prostatic Hyperplasia)”, published by Jiho Shuppan, 2001, pp. 11-24.

### DISCLOSURE OF THE INVENTION

### Problem to be Solved by the Invention

The object of the present invention is to provide an agent which can prevent the transition of benign prostatic hyperplasia to a surgical therapy.

### Means of Solving the Problems

Taking the above problems into consideration, the present inventors have conducted intensive studies and surprisingly found that by administering an indoline derivative represented by the above general formula (I) or a pharmaceutically acceptable salt thereof, transition to the surgical therapy can be prevented in patients with benign prostatic hyperplasia who are subject to a surgical therapy, thereby forming the basis of the present invention.

That is, the present invention relates to,

[1] a pharmaceutical composition for the prevention of transition to surgical therapy for benign prostatic hyperplasia, which comprises an indoline derivative represented by a general formula:

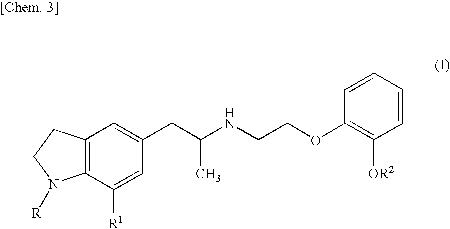

in the formula, R represents an aliphatic acyl group which may have one or more of a halogen atom, a hydroxyl group, a lower alkoxy group, a carboxyl group, a lower alkoxy-carbonyl group, a cycloalkyl group or an aryl group as a substituent group and may have an unsaturated bond in some cases, a hydroxyalkyl group, an aliphatic acyloxyalkyl group, a lower alkyl group which has a lower alkoxy group, a carboxy group, a lower alkoxycarbonyl group, an aryl-substituted lower alkoxycarbonyl group, a carbamoyl group, a mono or dialkyl-substituted carbamoyl group or a cyano group as a substituent group, an aromatic acyl group which may have one or more halogen atoms as a substituent group, a furoyl group or a pyridylcarbonyl group; R¹ represents a cyano group or a carbamoyl group; and R² represents a lower alkyl group which may have one or more of a halogen atom, a cyano group or an aryl group as a substituent group, or a pharmaceutically acceptable salt thereof as an active ingredient;

[2] a pharmaceutical composition for the prevention of transition to surgical therapy for benign prostatic hyperplasia as described in the above [1], wherein the indoline derivative is silodosin;

[3] a pharmaceutical composition for the prevention of transition to surgical therapy for benign prostatic hyperplasia as described in the above [1] or the above [2], wherein the surgical therapy is transurethral resection of the prostate;

[4] a pharmaceutical composition for the prevention of transition to surgical therapy for benign prostatic hyperplasia as described in any one of the above [1] to [3], which comprises administering to a patient who is subject to surgical therapy;

[5] a pharmaceutical composition for the prevention of transition to surgical therapy for benign prostatic hyperplasia as described in any one of the above [1] to [4], wherein daily dose of the indoline derivative represented by the above general formula (I) or a pharmaceutically acceptable salt thereof is from 2 to 16 mg;

[6] a pharmaceutical composition for treating a patient of benign prostatic hyperplasia whose overall severity is moderate or more, which comprises an indoline derivative represented by the above general formula (I) or a pharmaceutically acceptable salt thereof;

[7] a pharmaceutical composition for treating a patient of benign prostatic hyperplasia whose overall severity is moderate or more as described in the above [6], wherein the indoline derivative is silodosin;

[8] a pharmaceutical composition for treating a patient of benign prostatic hyperplasia whose overall severity is moderate or more as described in the above [6] or [7], wherein daily dose of the indoline derivative represented by the above general formula (I) or a pharmaceutically acceptable salt thereof is from 2 to 16 mg;

[9] a method for the prevention of transition to surgical therapy for benign prostatic hyperplasia, which comprises administering an effective amount of an indoline derivative represented by the above general formula (I) or a pharmaceutically acceptable salt thereof;

[10] a method for the prevention of transition to surgical therapy for benign prostatic hyperplasia as described in the above [9], wherein the indoline derivative is silodosin;

[11] a method for the prevention of transition to surgical therapy for benign prostatic hyperplasia as described in the above [9] or [10], wherein the surgical therapy is transurethral resection of the prostate;

[12] a method for the prevention of transition to surgical therapy for benign prostatic hyperplasia as described in the above [9] or [10], which comprises administering to a patient who is subject to surgical therapy;

[13] a method for the prevention of transition to surgical therapy for benign prostatic hyperplasia as described in the above [9] or [10], wherein daily dose of the indoline derivative represented by the above general formula (I) or a pharmaceutically acceptable salt thereof is from 2 to 16 mg;

[14] a method for treating a patient of benign prostatic hyperplasia whose overall severity is moderate or more, which comprises administering an effective amount of an indoline derivative represented by the above general formula (I) or a pharmaceutically acceptable salt thereof;

[15] a method for treating a patient of benign prostatic hyperplasia whose overall severity is moderate or more described in the above [14], wherein the indoline derivative is silodosin;

[16] a method for treating a patient of benign prostatic hyperplasia whose overall severity is moderate or more described in the above [14] or [15], wherein daily dose of the indoline derivative represented by the above general formula (I) or a pharmaceutically acceptable salt thereof is from 2 to 16 mg;

[17] a use of an indoline derivative represented by the above general formula (I) or a pharmaceutically acceptable salt thereof, for manufacturing a pharmaceutical composition for preventing the transition to surgical therapy for benign prostatic hyperplasia or for treating a patient of benign prostatic hyperplasia whose overall severity is moderate or more;

[18] a use described in the above [17], wherein the indoline derivative to be used is silodosin;

[19] a use described in the above [17] or [18], wherein daily dose of the indoline derivative to be used is from 2 to 16 mg; and the like.

### Effect of the Invention

The pharmaceutical composition which comprises an indoline derivative represented by the general formula (T) or a pharmaceutically acceptable salt thereof is useful as an agent for the prevention of transition to surgical therapy for a patient of benign prostatic hyperplasia who is subject to surgical therapy.

### Best Mode to Practice the Invention

In the above general formula (I), the term “lower alkyl” means a straight-chained or branched alkyl having from 1 to 6 carbon atoms, the term “hydroxyalkyl” means a straight-chained or branched alkyl having from 2 to 6 carbon atoms and having a hydroxyl group, with the proviso that said hydroxyl group is present at a position other than the α-position, the term “lower alkoxy” means a straight-chained or branched alkoxy having from 1 to 6 carbon atoms, and the term “cycloalkyl” means a 5- to 7-membered cyclic alkyl, respectively. In addition, the term “aryl” means an aromatic hydrocarbon such as phenyl, naphthyl or the like, the term “aromatic acyl” means acyl of a carboxylic acid having aryl which has the same meaning as defined above, the term “aliphatic acyl which may have an unsaturated bond” means acyl of a straight-chained or branched alkylcarboxylic acid having from 2 to 7 carbon atoms or a straight-chained or branched alkenylcarboxylic acid having from 3 to 7 carbon atoms, and the term “aliphatic acyloxy” means alkylcarbonyloxyalkyl having from 4 to 13 carbon atoms and having a hydroxyl group substituted with the above aliphatic acyl group, with the proviso that said aliphatic acyloxy group is present at a position other than the a-position, respectively. In addition, the term “furoyl” means 2-furoyl or 3-furoyl, the term “pyridylcarbonyl” means 2-pyridylcarbonyl, 3-pyridylcarbonyl or 4-pyridylcarbonyl, and the term “halogen atom” means a fluorine atom, a chlorine atom or a bromine atom, respectively. In this connection, the indoline derivative of the above general formula (I) can be prepared by the method as described in Patent Reference 2, and as said indoline derivatives, the above-mentioned silodosin, namely (−)-1-(3-hydroxypropyl)-5-((2R)-2-{[2-({2-[(2,2,2-trifluoroethyl)oxy]phenyl}oxy)ethyl]amino}propyl)-2,3-dihydro-1H-indole-7-carboxamide, is preferable.

In the above indoline derivatives represented by general formula (I), solvates with pharmaceutically acceptable solvents such as water, ethanol and the like are also included.

As the pharmaceutically acceptable salts of the above indoline derivative, for example, a compound having a carboxy group may be converted into its salt with an inorganic base such as sodium, potassium, calcium or the like or with an organic amine such as morpholine, piperidine or the like. Also, among the indoline derivatives, a compound in which the substituent group R is a substituted Or unsubstituted acyl group or a furoyl group may be converted into its monoacid addition salt with hydrochloric acid, hydrobromic acid, sulfuric acid, methanesulfonic acid, benzenesulfonic acid, p-toluene-sulfonic acid, acetic acid, citric acid, succinic acid, tartaric acid, 2,4-dimethylbenzenesulfonic acid, 2,4,6-trimethylbenzenesulfonic acid, (+)-camphorsulfonic acid, (−)-camphorsulfonic acid, 4-chlorobenzenesulfonic acid, 2-naphthalenesulfonic acid, 1-butanesulfonic acid, fumaric acid, giutamic acid, aspartic acid or the like. In addition, among the indoline derivatives, a compound in which the substituent group R is a substituted alkyl group or a pyridylcarbonyl group may be converted into its monoacid addition salt with hydrochloric acid, hydrobromic acid, sulfuric acid, methanesulfonic acid, p-toluene-sulfonic acid, 2,4-dimethylbenzenesulfonic acid, 2,5-dimethylbenzenesulfonic acid, 2,4,6-trimethylbenzene-sulfonic acid, (+)-camphorsulfonic acid, (−)-camphor-sulfonic acid, 4-chlorobenzenesulfonic acid, 2-naphthalene-sulfonic acid, 1-butanesulfonic acid, fumaric acid, glutamic acid, aspartic acid or the like.

The pharmaceutical composition of the present invention for the prevention of transition to surgical therapy for benign prostatic hyperplasia can be prepared by mixing the indoline derivative represented by the above general formula (I) or a pharmaceutically acceptable salt thereof with commonly used drug preparation carriers.

The drug preparation carriers may be used by optionally combining them depending on administration forms, and their examples include excipients such as lactose and the like; lubricants such as magnesium stearate and the like; disintegrating agents such as carboxymethylcellulose and the like; binders such as hydroxypropylmethylcellulose and the like; surfactants such as macrogol and the like; foaming agents such as sodium bicarbonate and the like; dissolving aids such as cyclodextrin and the like; acidity agents such as citric acid and the like; stabilizing agents such as sodium edetate and the like; pH adjusting agents such as phosphate and the like, and the like.

As the administration form of the pharmaceutical composition of the present invention for the prevention of transition to surgical therapy for benign prostatic hyperplasia, for example, oral administration preparations such as powders, granules, fine subtilaes, dry syrups, tablets, capsules and the like; parenteral administration preparations such as injections, patches, suppositories and the like, and the like can be illustrated, and oral administration preparations are preferable.

It is preferable to prepare the above preparations in such a manner that the indoline derivative represented by the above general formula (I) or a pharmaceutically acceptable salt thereof is administered within the range of from 2 to 16 mg, particularly from 4 to 8 mg, per day per adult, as the oral administration preparations.

In the present invention, the patients of benign prostatic hyperplasia who become the object of indication are patients with a severity which becomes the indication object of surgical therapy, namely moderate or more, excluding those patients who have urinary retention or complications caused by benign prostatic hyperplasia, and for example, patients of moderate or more based on the criteria for judging overall severity of the above guideline on the medical examination and treatment of benign prostatic hyperplasia can be illustrated, and patients of severe are preferable.

In the present invention, the surgical therapy for benign prostatic hyperplasia means an invasive treatment which is carried out with the aim of treating benign prostatic hyperplasia, and a low invasive treatment is also included therein. For example, open prostatectomy, transurethral resection of the prostate (TUR-P), thermotherapy, laser therapy, stent indwelling method and the like can be illustrated, and TUR-P is particularly preferable.

In the present invention, the prevention of transition to surgical therapy means a therapy which is carried out with the aim of avoiding the surgical therapy, or prolonging operating time of the surgical therapy, in the above patients who are subject to the surgical therapy.

### EXAMPLES

The present invention is further illustrated in more detail by way of the following Example. However, the present invention is not limited thereto.

### Example 1

Clinical effects in patients who are subject to surgical therapy for benign prostatic hyperplasia

Based on the results of a placebo-controlled, parallel groups-comparing double blind trial carried out in patients of benign prostatic hyperplasia, clinical effects of silodosin and tamsulosin hydrochloride in patients who are subject to surgical therapy were evaluated. As the patients who are subject to surgical therapy, patients whose overall severity before administration was “severe” based on the criteria for judging overall severity (Non-patent Reference 3) were extracted.

Objects: 147 patients who are subject to surgical therapy for benign prostatic hyperplasia

Administration method: oral administration for 12 weeks

Administered groups: Silodosin group (4 mg silodosin/once, twice a day, 63 patients), Tamsulosin group (0.2 mg tamsulosin hydrochloride, once a day, 55 patients) and Placebo group (placebo drug, 29 patients).

Primary endpoints: I-PSS total score, QOL score

Criteria for judging overall severity: in the severity of respective judging items shown in Table 1, the overall severity is regarded as “severe” when “severe” is present in 2 items or more. In this connection, the Qmax in the table means maximum flow rate (mL/second), RU means residual urine volume (mL) and PV means estimated prostate volume (mL) by ultrasonic tomography.

TABLE 1
Criteria for judging overall severity

| Respective judging | | |
|---|---|---|
| items | Moderate | Severe |
| 1. I-PSS total | 8-19 | 20-35 |
| score | | |
| 2. QOL score | 2, 3, 4 | 5, 6 |
| 3. Qmax, Residual | Qmax: 5 mL/sec or more | Qmax: less than 5 mL/ |
| urine volume | and RU: less than 100 mL | sec or RU: 100 mL or |
| | | more |
| 4. Estimated | 20 mL or more, and less | 50 mL or more |
| prostate volume | than 50 mL | |
| (PV) | | |

I-PSS items questioned
(1) Sensation of residual urine

“Have you had a sensation of not emptying your bladder completely after you finished urinating?”
(2) Urination within 2 hours

“Have you had to urinate again less than two hours after you finished urinating?”
(3) Intermittency of urinary stream

“Have you found you stopped and started again several times when you urinated?”
(4) Urgency

“Have you found it difficult to postpone urination?”
(5) Power of urinary stream

“Have you had a weak urinary stream?”
(6) Straining during urination

“Have you had to push or strain to begin urination?”

The scores of (1) to (6) are as follows.

Not at all: 0 point, less than 1 time in 5: 1 point, less than 1 time in 2: 2 points, about 1 time in 2: 3 points, more than 1 time in 2: 4 points, almost always: 5 points.
(7) Nocturia

“How many times did you get up to urinate from the time you went to bet at night until the time you got up in the morning?”

The scores are as follows.

None: 0 point, 1 time: 1 point, 2 times: 2 points, 3 times: 3 points, 4 times: 4 points, 5 times or more: 5 points.

QOL score

“How do you feel if the present condition of urination will continue from now on?”

Very satisfied 0 point, satisfied: 1 point, generally satisfied: 2 points, neither satisfied nor unsatisfied: 3 points, a little unsatisfied: 4 points, unsatisfied: 5 points, very unsatisfied: 6 points.

Results

Regarding the case of patients whose overall severity before administration was severe based on the criteria for judging overall severity (Non-patent Reference 3), changes in I-PSS total score and QOL score at the final evaluation were compared. The results are shown in Table 2 and Table 3, respectively. In this connection, comparison between the administration groups was carried out by two sample t-test, and in the tables, “*” and “**” show that significant differences were found at significance levels of less than 5% and less than 1%, respectively, and “N.S.” means that a significance was not found.

TABLE 2
Change in I-PSS total score

| | | | Change in I-PSS at |
|---|---|---|---|
| | | | the end |
| Administration | | I-PSS before | Mean ± standard |
| groups | Cases | administration | deviation |
| Silodosin | 63 | 22.8 | −12.0 ± 7.1¹⁾ *²⁾ ** |
| Tamsulosin | 55 | 21.9 | −8.6 ± 6.2^{3) N.S.} |
| Placebo | 29 | 23.5 | −8.2 ± 8.2 |

¹⁾Silodosin group vs. Placebo group
²⁾Silodosin group vs. Tamsulosin group
³⁾Tamsulosin group vs. Placebo group
*: P < 0.05;
**: P < 0.01;
N.S.: no significance

TABLE 3
Change in QOL score

| | | | Change in QOL at |
|---|---|---|---|
| Administration | | QOL before | the end |
| groups | Cases | administration | Mean ± SD |
| Silodosin | 63 | 5.5 | −2.3 ± 1.6¹⁾ ** ²⁾ ** |
| Tamsulosin | 55 | 5.3 | −1.5 ± 1.3^{3) N.S.} |
| Placebo | 29 | 5.3 | −1.4 ± 1.3 |

¹⁾Silodosin group vs. Placebo group
²⁾Silodosin group vs. Tamsulosin group
³⁾Tamsulosin group vs. Placebo group
**: P < 0.01;
N.S.: no significance

As shown in Table 2, Silodosin group significantly improved the I-PSS total score in the patients of benign prostatic hyperplasia whose overall severity before administration was severe. The effect of Silodosin group was statistically significant in comparison with Placebo group and Tamsulosin group. In addition, as shown in Table 3, Silodosin group also improved QOL score statistically significantly in comparison with Placebo group and Tamsulosin group. Based on this, it was shown that silodosin significantly improves I-PSS total score in patients of benign prostatic hyperplasia who are subject to surgical therapy and has an effect to avoid transition to surgical therapy.

Next, the effect of each administration group to improve the severity of I-PSS total score was evaluated in the patients of benign prostatic hyperplasia whose overall severity before administration was severe. The results are shown in Table 4. In this connection, comparison between the administration groups was carried out by two sample Wilcoxon test, and in the table, “*” and “**” show that significant differences were found at significance levels of less than 5% and less than 1%, respectively, and “N.S.” means that a significance was not found.

TABLE 4
Improving effect on severity of I-PSS total score

| | | Severity of subjective | | | |
|---|---|---|---|---|---|
| | | symptoms at the end. | | | |
| | | Number of patients | | | |
| | | (%) | | | |
| Administration | | Mild | Moderate | Severe | Comparison |
| groups | Cases | (0-7) | (8-19) | (20-35) | between groups |
| Silodosin | 63 | 24 | 33 | 6 | 1) ** |
| | | (38.1%) | (52.4%) | (9.5%) | 2) * |
| Tamsulosin | 55 | 12 | 34 | 9 | 3) N.S. |
| | | (21.8%) | (61.8%) | (16.4%) | |
| Placebo | 29 | 6 | 11 | 12 | — |
| | | (20.7%) | (37.9%) | (41.4%) | |

1) Silodosin group vs. Placebo group
2) Silodosin group vs. Tamsulosin group
3) Tamsulosin group vs. Placebo group
*: P < 0.05;
**: P < 0.01;
N.S.; no significance

As shown in Table 4, Silodosin group significantly improved the severity of I-PSS total score in the patients of benign prostatic hyperplasia whose overall severity before administration was severe. That is, it can be seen that the severity of I-PSS total score in about 38% of the patients who were subject to surgical therapy was improved to mild. The improving effect of Silodosin group was a statistically significant deference in comparison with Placebo group and TamsulQsin group.

As shown above, in the placebo-controlled, double blind, comparative clinical test, silodosin was able to significantly improve I-PSS total score and QOL score of the patients of benign prostatic hyperplasia whose overall severity before administration was severe and to improve symptoms of the patients who are subject to surgical therapy to a level outside of surgical therapy object, and thereby to avoid transition to surgical therapy.

### Example 2

Effects on the objective symptoms of patients who are subject to surgical therapy for benign prostatic hyperplasia

Based on the results from a long-term administration test in patients of benign prostatic hyperplasia, effects of silodosin on objective symptoms of patients who are subject to surgical therapy were examined. As the patients who are subject to surgical therapy, patients whose overall severities before administration were “moderate” and “severe” according to the criteria for judging overall severity described in the above Table 1 were extracted.

Objects: 229 patients who are subject to surgical therapy for benign prostatic, hyperplasia

Administration method: 4 mg silodosin/once (can be optionally reduced to 2 mg/once), twice a day, oral administration for 52 weeks

Analyzed groups: 101 severe patients (reduced to 2 mg/once in 9 cases among them), 128 moderate patients (reduced to 2 mg/once in 17 cases among them)

As an objective symptom, results of maximum flow rate (Qmax) (mL/sec) are shown in Table 5

TABLE 5
Improving effect on maximum flow rate (Qmax)

| | | | Before | Change after |
|---|---|---|---|---|
| | Administration | | administration | 52 weeks |
| Group | group | Cases | (mean ± SD) | (mean ± SD) |
| Severe | Silodosin | 101 | 8.9 ± 3.0 | 3.2 ± 4.9 |
| Moderate | Silodosin | 128 | 10.3 ± 3.0 | 2.3 ± 5.3 |

As shown in Table 5, the changes after 52 weeks of silodosin administration were 3.2 mL/sec in the severe group and 2.3 mL/sec in the moderate group, and the improvement of the objective symptom was observed in each group in comparison with the case of before administration. Particularly, the effect was significant in the severe group.

### INDUSTRIAL APPLICABILITY

The pharmaceutical composition of the invention is markedly useful as an agent for the prevention of transition to surgical therapy, because it can significantly improve subjective and objective symptoms of patients who are subject to surgical therapy for benign prostatic hyperplasia.

1. A pharmaceutical composition for the prevention of transition to surgical therapy for benign prostatic hyperplasia, which comprises an indoline derivative represented by a general formula (I): 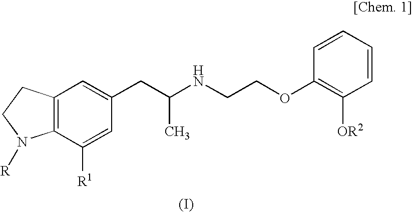
in the formula, R represents an aliphatic acyl group which may have one or more of a halogen atom, a hydroxyl group, a lower alkoxy group, a carboxyl group, a lower alkoxycarbonyl group, a cycloalkyl group or an aryl group as a substituent group and may have an unsaturated bond in some cases, a hydroxyalkyl group, an aliphatic acyloxyalkyl group, a lower alkyl group which has a lower alkoxy group, a carboxy group, a lower alkoxycarbonyl group, an aryl-substituted lower alkoxycarbonyl group, a carbamoyl group, a mono or dialkyl-substituted carbamoyl group or a cyano group as a substituent group, an aromatic acyl group which may have one or more halogen atoms as a substituent group, a furoyl group or a pyridylcarbonyl group; R¹ represents a cyano group or a carbamoyl group; and R² represents a lower alkyl group which may have one or more of a halogen atom, a cyano group or an aryl group as a substituent group, or a pharmaceutically acceptable salt thereof.

2. A pharmaceutical composition for the prevention of transition to surgical therapy for benign prostatic hyperplasia as claimed in claim 1, wherein the indoline derivative is silodosin.

3. A pharmaceutical composition for the prevention of transition to surgical therapy for benign prostatic hyperplasia as claimed in claim 1 or 2, wherein the surgical therapy is transurethral resection of the prostate.

4. A pharmaceutical composition for the prevention of transition to surgical therapy for benign prostatic hyperplasia as claimed in any one of claims 1, which comprises administering to a patient who is subject to surgical therapy.

5. A pharmaceutical composition for the prevention of transition to surgical therapy for benign prostatic hyperplasia as claimed in any one of claims 1, wherein daily dose of the indoline derivative represented by the general formula (I) or a pharmaceutically acceptable salt thereof is from 2 to 16 mg.

6. A pharmaceutical composition for treating a patient of benign prostatic hyperplasia whose overall severity is moderate or more, which comprises an indoline derivative represented by a general formula (I): 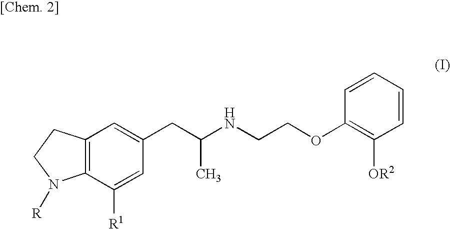
in the formula, R represents an aliphatic acyl group which may have one or more of a halogen atom, a hydroxyl group, a lower alkoxy group, a carboxyl group, a lower alkoxycarbonyl group, a cycloalkyl group or an aryl group as a substituent group and may have an unsaturated bond in some cases, a hydroxyalkyl group, an aliphatic acyloxyalkyl group, a lower alkyl group which has a lower alkoxy group, a carboxy group, a lower alkoxycarbonyl group, an aryl-substituted lower alkoxycarbonyl group, a carbamoyl group, a mono or dialkyl-substituted carbamoyl group or a cyano group as a substituent group, an aromatic acyl group which may have one or more halogen atoms as a substituent group, a furoyl group or a pyridylcarbonyl group; R¹ represents a cyano group or a carbamoyl group; and R² represents a lower alkyl group which may have one or more of a halogen atom, a cyano group or an aryl group as a substituent group, or a pharmaceutically acceptable salt thereof.

7. A pharmaceutical composition for treating a patient of benign prostatic hyperplasia whose overall severity is moderate or more as claimed in claim 6, wherein the indoline derivative is silodosin.

8. A pharmaceutical composition for treating a patient of benign prostatic hyperplasia whose overall severity is moderate or more as claimed in claim 6 or 7, wherein daily dose of the indoline derivative represented by the general formula (I) or a pharmaceutically acceptable salt thereof is from 2 to 16 mg.

9. A method for the prevention of transition to surgical therapy for benign prostatic hyperplasia, which comprises administering an effective amount of an indoline derivative represented by a general formula (I): 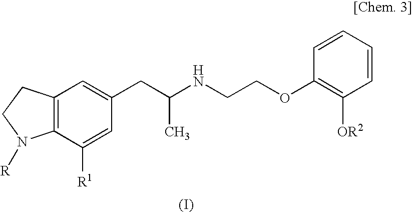
in the formula, R represents an aliphatic acyl group which may have one or more of a halogen atom, a hydroxyl group, a lower alkoxy group, a carboxyl group, a lower alkoxycarbonyl group, a cycloalkyl group or an aryl group as a substituent group and may have an unsaturated bond in some cases, a hydroxyalkyl group, an aliphatic acyloxyalkyl group, a lower alkyl group which has a lower alkoxy group, a carboxy group, a lower alkoxycarbonyl group, an aryl-substituted lower alkoxycarbonyl group, a carbamoyl group, a mono or dialkyl-substituted carbamoyl group or a cyano group as a substituent group, an aromatic acyl group which may have one or more halogen atoms as a substituent group, a furoyl group or a pyridylcarbonyl group; R¹ represents a cyano group or a carbamoyl group; and R² represents a lower alkyl group which may have one or more of a halogen atom, a cyano group or an aryl group as a substituent group, or a pharmaceutically acceptable salt thereof.

10. A method for the prevention of transition to surgical therapy for benign prostatic hyperplasia as claimed in claim 9, wherein the indoline derivative is silodosin.

11. A method for the prevention of transition to surgical therapy for benign prostatic hyperplasia as claimed in claim 9 or 10, wherein the surgical therapy is transurethral resection of the prostate.

12. A method for the prevention of transition to surgical therapy for benign prostatic hyperplasia as claimed in claim 9 or 10, which comprises administering to a patient who is subject to surgical therapy.

13. A method for the prevention of transition to surgical therapy for benign prostatic hyperplasia as claimed in claim 9 or 10, wherein daily dose of the indoline derivative represented by the general formula (I) or a pharmaceutically acceptable salt thereof is from 2 to 16 mg.

14. A method for treating a patient of benign prostatic hyperplasia whose overall severity is moderate or more, which comprises administering an effective amount of an indoline derivative represented by a general formula (I): 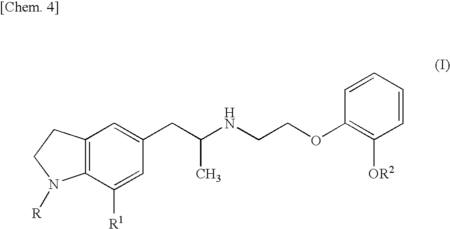
in the formula, R represents an aliphatic acyl group which may have one or more of a halogen atom, a hydroxyl group, a lower alkoxy group, a carboxyl group, a lower alkoxycarbonyl group, a cycloalkyl group or an aryl group as a substituent group and may have an unsaturated bond in some cases, a hydroxyalkyl group, an aliphatic acyloxyalkyl group, a lower alkyl group which has a lower alkoxy group, a carboxy group, a lower alkoxycarbonyl group, an aryl-substituted lower alkoxycarbonyl group, a carbamoyl group, a mono or dialkyl-substituted carbamoyl group or a cyano group as a substituent group, an aromatic acyl group which may have one or more halogen atom as a substituent group, a furoyl group or a pyridylcarbonyl group; R¹ represents a cyano group or a carbamoyl group; and R² represents a lower alkyl group which may have one or more of a halogen atom, a cyano group or an aryl group as a substituent group, or a pharmaceutically acceptable salt thereof.

15. A method for treating a patient of benign prostatic hyperplasia whose overall severity is moderate or more as claimed in claim 14, wherein the indoline derivative is silodosin.

16. A method for treating a patient of benign prostatic hyperplasia whose overall severity is moderate or more as claimed in claim 14 or 15, wherein daily dose of the indoline derivative represented by the general formula (I) or a pharmaceutically acceptable salt thereof is from 2 to 16 mg.

17-19. (canceled)
